(19) United States
(12) Patent Application Publication
(10) Pub. No.: US 2019/0321647 A1
(43) Pub. Date: Oct. 24, 2019

(54) WEARABLE CARDIOVERTER DEFIBRILLATOR (WCD) SYSTEM WITH THERAPY DIVERT BUTTON THAT IS OVERRIDABLE, FOR EXAMPLE WHEN ACCIDENTALLY PRESSED CONTINUOUSLY

(71) Applicant: West Affum Holdings Corp., Grand Cayman (KY)

(72) Inventors: Zoie Engman, Kirkland, WA (US); Pamela Breske, Newcastle, WA (US); David Finch, Bothell, WA (US); Erick Roane, Bellevue, WA (US); Aaron Piazza, Lake Forest Park, WA (US)

(21) Appl. No.: 16/362,072

(22) Filed: Mar. 22, 2019

Related U.S. Application Data
(60) Provisional application No. 62/662,058, filed on Apr. 24, 2018.

(51) Int. Cl. A61N 1/39; A61N 1/04

(52) U.S. Cl. CPC A61N 1/3904; A61N 1/3987; A61B 5/046; A61N 1/0484; A61N 1/3925; A61N 1/046

(57) ABSTRACT

In embodiments, a wearable cardioverter defibrillator (WCD) system includes an electrode, a support structure configured to be worn by an ambulatory patient so as to maintain the electrode on the patient's body, and an energy storage module configured to store a charge that can be discharged via the electrode to deliver an electric shock to the patient. The WCD system further includes a divert actuator, for the patient to prevent an imminent discharge that is unnecessary. In some embodiments, actuating the divert actuator is overridden, for instance where it is inferred that the divert actuator is being actuated inadvertently. An advantage can be that a life-saving shock is thus not withheld. In some embodiments the divert actuator is used also for playing an instructional message, or for initiating long-term recording of an episode. An advantage can be that the patient needs to learn about fewer controls of the WCD system.

### CROSS REFERENCE TO RELATED PATENT APPLICATIONS

This patent application claims priority from U.S. Provisional Patent Application Ser. No. 62/662,058, filed on Apr. 24, 2018.

### BACKGROUND

When people suffer from some types of heart arrhythmias, the result may be that blood flow to various parts of the body is reduced. Some arrhythmias may even result in a Sudden Cardiac Arrest (SCA). SCA can lead to death very quickly, e.g. within 10 minutes, unless treated in the interim. Some observers have thought that SCA is the same as a heart attack, which it is not.

Some people have an increased risk of SCA. Such people include patients who have had a heart attack, or a prior SCA episode. A frequent recommendation for these people is to receive an Implantable Cardioverter Defibrillator (ICD). The ICD is surgically implanted in the chest, and continuously monitors the patient's electrocardiogram (ECG). If certain types of heart arrhythmias are detected, then the ICD delivers an electric shock through the heart.

As a further precaution, people who have been identified to have an increased risk of an SCA are sometimes given a Wearable Cardioverter Defibrillator (WCD) system, to wear until the time that their ICD is implanted. Early versions of such systems were called wearable cardiac defibrillator systems. A WCD system typically includes a harness, vest, belt, or other garment that the patient is to wear. The WCD system further includes electronic components, such as a defibrillator and electrodes, coupled to the harness, vest, or other garment. When the patient wears the WCD system, the electrodes may make good electrical contact with the patient's skin, and therefore can help sense the patient's ECG. If a shockable heart arrhythmia is detected from the ECG, then the defibrillator delivers an appropriate electric shock through the patient's body, and thus through the heart. This may restart the patient's heart and thus save their life.

All subject matter discussed in this Background section of this document is not necessarily prior art, and may not be presumed to be prior art simply because it is presented in this Background section. Plus, any reference to any prior art in this description is not, and should not be taken as, an acknowledgement or any form of suggestion that such prior art forms parts of the common general knowledge in any art in any country. Along these lines, any recognition of problems in the prior art discussed in this Background section or associated with such subject matter should not be treated as prior art, unless expressly stated to be prior art. Rather, the discussion of any subject matter in this Background section should be treated as part of the approach taken towards the particular problem by the inventors. This approach in and of itself may also be inventive.

### BRIEF SUMMARY

The present description gives instances of wearable cardioverter defibrillator (WCD) systems, storage media that may store programs, and methods, the use of which may help overcome problems and limitations of the prior art.

In embodiments, a wearable cardioverter defibrillator (WCD) system includes an electrode, a support structure configured to be worn by an ambulatory patient so as to maintain the electrode on the patient's body, and an energy storage module configured to store a charge that can be discharged via the electrode to deliver an electric shock to the patient. The WCD system further includes a divert actuator, for the patient to prevent an imminent discharge that is unnecessary. In some embodiments, actuating the divert actuator is overridden, for instance where it is inferred that the divert actuator is being actuated inadvertently. An advantage can be that a life-saving shock is thus not withheld. In some embodiments the divert actuator is used also for playing an instructional message, or for initiating long-term recording of an episode. An advantage can be that the patient needs to learn about fewer controls of the WCD system.

These and other features and advantages of the claimed invention will become more readily apparent in view of the embodiments described and illustrated in this specification, namely in this written specification and the associated drawings.

### BRIEF DESCRIPTION OF THE DRAWINGS

FIG. 1 is a diagram of components of a sample wearable cardioverter defibrillator (WCD) system, made according to embodiments.

FIG. 2 is a diagram showing sample components of an external defibrillator, such as the one belonging in the system of FIG. 1, and which is made according to embodiments.

FIG. 3 is a diagram of sample embodiments of components of a WCD system.

FIG. 4 is a flowchart for illustrating a method about a therapy divert button of a WCD system in the prior art.

FIG. 5 is a conceptual diagram for illustrating that a divert actuator may be subject to an override condition according to embodiments.

FIG. 6 is a flowchart for illustrating sample methods for a WCD system where actuating a divert actuator may be overridden according to embodiments.

FIG. 7 is a sample state diagram for a processor of a WCD system, where the processor alternates among being in one of a plurality of possible states, according to embodiments.

FIG. 8 is a flowchart for illustrating sample methods according to embodiments.

FIG. 9 is a flowchart for illustrating sample methods for a WCD system where an instructional message may be played according to embodiments.

FIG. 10 shows time diagrams resulting from embodiments where a divert actuator is used by the patient to initiate a long term-recording of a patient input, such as for a patient episode.

FIG. 11 is a flowchart for illustrating sample methods for a WCD system where a long term-recording of a patient input during an episode may be initiated by the patient according to embodiments.

### DETAILED DESCRIPTION

As has been mentioned, the present description is about wearable cardioverter defibrillator (WCD) systems, storage media that may store programs, and methods. Embodiments are now described in more detail.

A wearable cardioverter defibrillator (WCD) system according to embodiments may protect an ambulatory patient by electrically restarting their heart if needed. Such a WCD system may have a number of components. These components can be provided separately as modules that can be interconnected, or can be combined with other components, and so on.

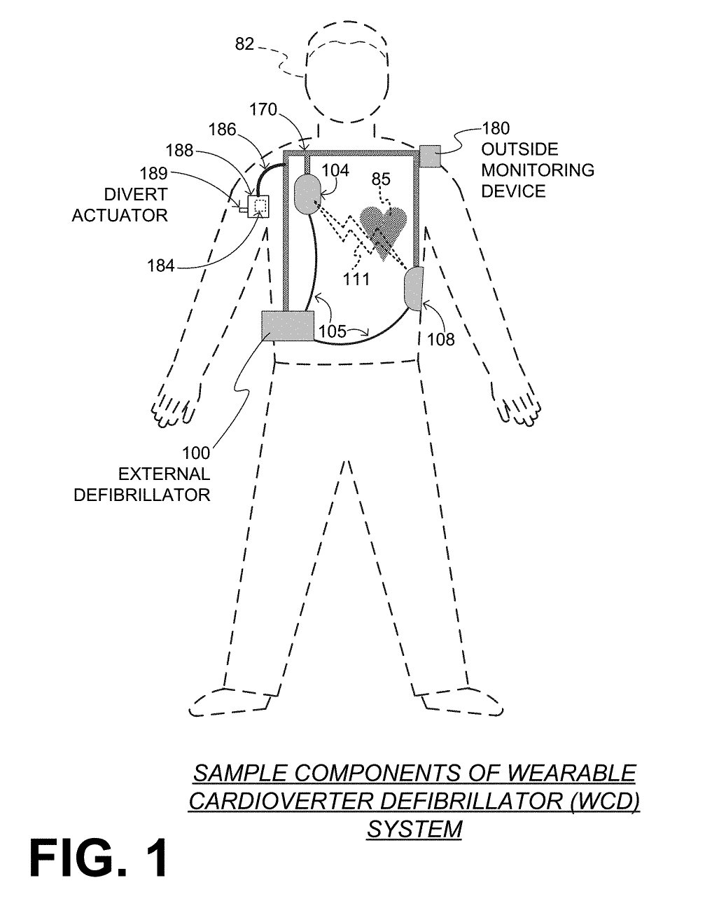

FIG. 1 depicts a patient 82. Patient 82 may also be referred to as a person and/or wearer, since the patient is wearing components of the WCD system. Patient 82 is ambulatory, which means that, while wearing the wearable portion of the WCD system, patient 82 can walk around and is not necessarily bed-ridden. While patient 82 may be considered to be also a “user” of the WCD system, this is not a requirement. For instance, a user of the wearable cardioverter defibrillator (WCD) may also be a clinician such as a doctor, nurse, emergency medical technician (EMT) or other similarly tasked individual or group of individuals. In some cases, a user may even be a bystander. The particular context of these and other related terms within this description should be interpreted accordingly.

A WCD system according to embodiments can be configured to defibrillate the patient who is wearing the designated parts the WCD system. Defibrillating can be by the WCD system delivering an electrical charge to the patient's body in the form of an electric shock. The electric shock can be delivered in one or more pulses.

In particular, FIG. 1 also depicts components of a WCD system made according to embodiments. One such component is a support structure 170 that is wearable by ambulatory patient 82. Accordingly, support structure 170 is configured to be worn by ambulatory patient 82 for at least several hours per day, and for at least several days, even a few months. It will be understood that support structure 170 is shown only generically in FIG. 1, and in fact partly conceptually. FIG. 1 is provided merely to illustrate concepts about support structure 170, and is not to be construed as limiting how support structure 170 is implemented, or how it is worn.

Support structure 170 can be implemented in many different ways. For example, it can be implemented in a single component or a combination of multiple components. In embodiments, support structure 170 could include a vest, a half-vest, a garment, etc. In such embodiments such items can be worn similarly to analogous articles of clothing. In embodiments, support structure 170 could include a harness, one or more belts or straps, etc. In such embodiments, such items can be worn by the patient around the torso, hips, over the shoulder, etc. In embodiments, support structure 170 can include a container or housing, which can even be waterproof. In such embodiments, the support structure can be worn by being attached to the patient's body by adhesive material, for example as shown and described in U.S. Pat. No. 8,024,037. Support structure 170 can even be implemented as described for the support structure of US Pat. App. No. US2017/0056682, which is incorporated herein by reference. Of course, in such embodiments, the person skilled in the art will recognize that additional components of the WCD system can be in the housing of a support structure instead of being attached externally to the support structure, for example as described in the US2017/0056682 document. There can be other examples.

FIG. 1 shows a sample external defibrillator 100. As described in more detail later in this document, some aspects of external defibrillator 100 include a housing and an energy storage module within the housing. As such, in the context of a WCD system, defibrillator 100 is sometimes called a main electronics module. The energy storage module can be configured to store an electrical charge. Other components can cause at least some of the stored electrical charge to be discharged via electrodes through the patient, so as to deliver one or more defibrillation shocks through the patient.

FIG. 1 also shows sample defibrillation electrodes 104, 108, which are coupled to external defibrillator 100 via electrode leads 105. Defibrillation electrodes 104, 108 can be configured to be worn by patient 82 in a number of ways. For instance, defibrillator 100 and defibrillation electrodes 104, 108 can be coupled to support structure 170, directly or indirectly. In other words, support structure 170 can be configured to be worn by ambulatory patient 82 so as to maintain at least one of electrodes 104, 108 on the body of ambulatory patient 82, while patient 82 is moving around, etc. The electrode can be thus maintained on the body by being attached to the skin of patient 82, simply pressed against the skin directly or through garments, etc. In some embodiments the electrode is not necessarily pressed against the skin, but becomes biased that way upon sensing a condition that could merit intervention by the WCD system. In addition, many of the components of defibrillator 100 can be considered coupled to support structure 170 directly, or indirectly via at least one of defibrillation electrodes 104, 108.

When defibrillation electrodes 104, 108 make good electrical contact with the body of patient 82, defibrillator 100 can administer, via electrodes 104, 108, a brief, strong electric pulse 111 through the body. Pulse 111 is also known as shock, defibrillation shock, therapy, electrotherapy, therapy shock, etc. Pulse 111 is intended to go through and restart heart 85, in an effort to save the life of patient 82. Pulse 111 can further include one or more pacing pulses of lesser magnitude to simply pace heart 85 if needed, and so on.

Sometimes a WCD system may diagnose incorrectly patient 82. In fact, such a WCD system may even administer defibrillation shock 111 as therapy to patient 82 when patient 82 does not need it, for instance when patient 82 is not having SCA, and is conscious. To prevent an undesirable shock 111 under such circumstances, a WCD system according to embodiments may further include a divert actuator 189 that is coupled to support structure 170 and configured to be actuated by patient 82. Divert actuator 189 is also known as cancel switch, “I am alive” switch, “live man” switch, therapy divert switch, and so on. As will be seen later in this document, divert actuator 189 is typically one more input device of an overall user interface 280.

In the shown embodiment, divert actuator 189 is a button in a stand-alone small housing 188 that has a wire 186. Wire 186 can, in turn, be ultimately electrically coupled with external defibrillator 100. In other embodiments, the divert actuator can be a lever, a switch, etc. In such embodiments, the WCD system may further have an output device configured to output an alarm, and permit patient 82 some time to actuate divert actuator 189 responsive to the alarm. If patient 82 does that within the permitted time, then patient 82 is not shocked.

In such embodiments, the WCD system may further include a tactile output device 184, for example within small housing 188. Device 184 can be configured to output a confirmation vibration, responsive to the divert actuator 189 being actuated. For example, responsive to patient 82 pushing button 189, a small motor can rotate briefly to produce the confirmation vibration.

A prior art defibrillator typically decides whether to defibrillate or not based on an ECG signal of the patient. However, external defibrillator 100 may initiate defibrillation, or hold-off defibrillation, based on a variety of inputs, with the ECG signal merely being one of these inputs.

A WCD system according to embodiments can obtain data from patient 82. For collecting such data, the WCD system may optionally include at least an outside monitoring device 180. Device 180 is called an “outside” device because it could be provided as a standalone device, for example not within the housing of defibrillator 100. Device 180 can be configured to sense or monitor at least one local parameter. A local parameter can be a parameter of patient 82, or a parameter of the WCD system, or a parameter of the environment, as will be described later in this document.

For some of these parameters, device 180 may include one or more sensors or transducers. Each one of such sensors can be configured to sense a parameter of patient 82, and to render an input responsive to the sensed parameter. In some embodiments the input is quantitative, such as values of a sensed parameter; in other embodiments the input is qualitative, such as informing whether or not a threshold is crossed, and so on. Sometimes these inputs about patient 82 are also called physiological inputs and patient inputs. In embodiments, a sensor can be construed more broadly, as encompassing many individual sensors.

Optionally, device 180 is physically coupled to support structure 170. In addition, device 180 may be communicatively coupled with other components that are coupled to support structure 170. Such communication can be implemented by a communication module, as will be deemed applicable by a person skilled in the art in view of this description.

In embodiments, one or more of the components of the shown WCD system may be customized for patient 82. This customization may include a number of aspects. For instance, support structure 170 can be fitted to the body of patient 82. For another instance, baseline physiological parameters of patient 82 can be measured, such as the heart rate of patient 82 while resting, while walking, motion detector outputs while walking, etc. The measured values of such baseline physiological parameters can be used to customize the WCD system, in order to make its diagnoses more accurate, since patients' bodies differ from one another. Of course, such parameter values can be stored in a memory of the WCD system, and so on. Moreover, a programming interface can be made according to embodiments, which receives such measured values of baseline physiological parameters. Such a programming interface may input automatically in the WCD system these, along with other data.

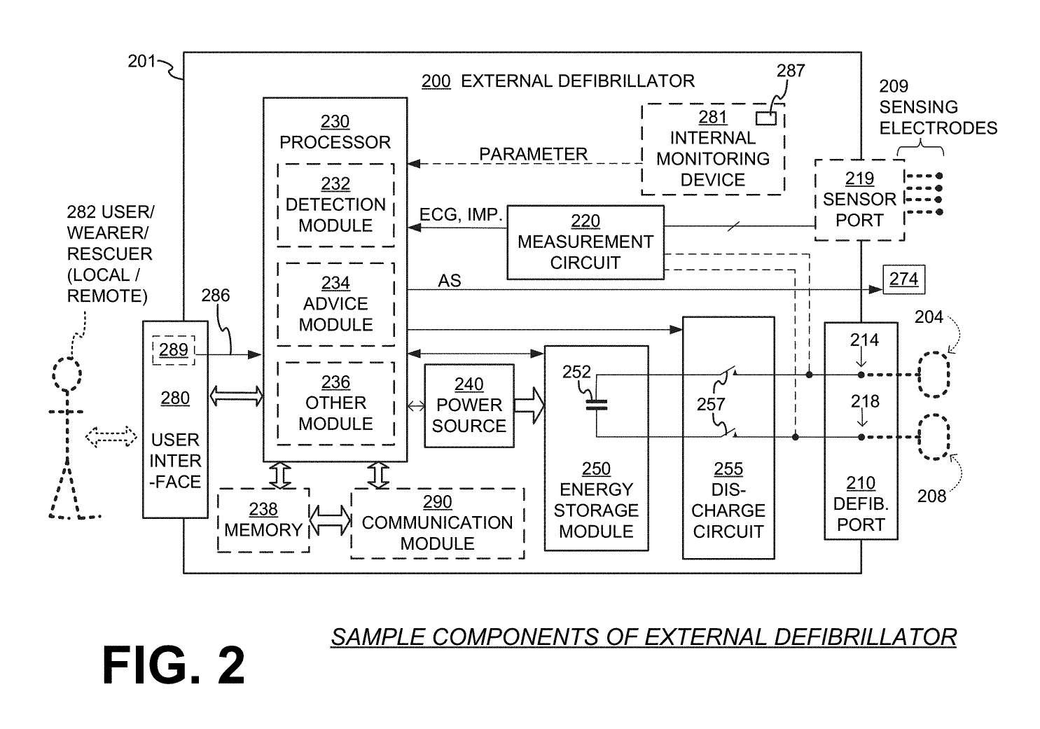

FIG. 2 is a diagram showing components of an external defibrillator 200, made according to embodiments. These components can be, for example, included in external defibrillator 100 of FIG. 1. The components shown in FIG. 2 can be provided in a housing 201, which may also be referred to as casing 201.

External defibrillator 200 is intended for a patient who would be wearing it, such as ambulatory patient 82 of FIG. 1. Defibrillator 200 may further include a user interface 280 for a user 282. User 282 can be patient 82, also known as wearer 82. Or, user 282 can be a local rescuer at the scene, such as a bystander who might offer assistance, or a trained person. Or, user 282 might be a remotely located trained caregiver in communication with the WCD system.

User interface 280 can be made in a number of ways. User interface 280 may include output devices, which can be visual, audible or tactile, for communicating to a user by outputting images, sounds or vibrations. Images, sounds, vibrations, and anything that can be perceived by user 282 can also be called human-perceptible indications (HPIs). There are many examples of output devices. For example, an output device can be a light, or a screen to display what is sensed, detected and/or measured, and provide visual feedback to rescuer 282 for their resuscitation attempts, and so on. Another output device can be a speaker, which can be configured to issue voice prompts, beeps, loud alarm sounds and/or words to warn bystanders, etc.

User interface 280 may further include input devices for receiving inputs from users. Such input devices may include various controls, such as pushbuttons, keyboards, touchscreens, one or more microphones, and so on. One such input device 289 can be a divert actuator, such as divert actuator 189. Input device 289 may generate a signal 286 for processor 230. The embodiment of FIG. 2 suggests that the divert actuator can be on the housing 201 of defibrillator 200, which is also possible.

Defibrillator 200 may include an internal monitoring device 281. Device 281 is called an “internal” device because it is incorporated within housing 201. Monitoring device 281 can sense or monitor patient parameters such as patient physiological parameters, system parameters and/or environmental parameters, all of which can be called patient data. In other words, internal monitoring device 281 can be complementary or an alternative to outside monitoring device 180 of FIG. 1. Allocating which of the parameters are to be monitored by which of monitoring devices 180, 281 can be done according to design considerations. Device 281 may include one or more sensors, as also described elsewhere in this document.

Patient parameters may include patient physiological parameters. Patient physiological parameters may include, for example and without limitation, those physiological parameters that can be of any help in detecting by the WCD system whether or not the patient is in need of a shock or other intervention or assistance. Patient physiological parameters may also optionally include the patient's medical history, event history and so on. Examples of such parameters include the patient's ECG, blood oxygen level, blood flow, blood pressure, blood perfusion, pulsatile change in light transmission or reflection properties of perfused tissue, heart sounds, heart wall motion, breathing sounds and pulse. Accordingly, monitoring devices 180, 281 may include one or more sensors configured to acquire patient physiological signals. Examples of such sensors or transducers include one or more electrodes to detect ECG data, a perfusion sensor, a pulse oximeter, a device for detecting blood flow (e.g. a Doppler device), a sensor for detecting blood pressure (e.g. a cuff), an optical sensor, illumination detectors and sensors perhaps working together with light sources for detecting color change in tissue, a motion sensor, a device that can detect heart wall movement, a sound sensor, a device with a microphone, an SpO₂ sensor, and so on. In view of this disclosure, it will be appreciated that such sensors can help detect the patient's pulse, and can therefore also be called pulse detection sensors, pulse sensors, and pulse rate sensors. In addition, a person skilled in the art may implement other ways of performing pulse detection.

In some embodiments, the local parameter is a trend that can be detected in a monitored physiological parameter of patient 282. A trend can be detected by comparing values of parameters at different times over short and long terms. Parameters whose detected trends can particularly help a cardiac rehabilitation program include: a) cardiac function (e.g. ejection fraction, stroke volume, cardiac output, etc.); b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO₂, CO₂, or other parameters such as those mentioned above, f) respiratory function, respiratory rate, etc.; g) motion, level of activity; and so on. Once a trend is detected, it can be stored and/or reported via a communication link, along perhaps with a warning if warranted. From the report, a physician monitoring the progress of patient 282 will know about a condition that is either not improving or deteriorating.

Patient state parameters include recorded aspects of patient 282, such as motion, posture, whether they have spoken recently plus maybe also what they said, and so on, plus optionally the history of these parameters. Or, one of these monitoring devices could include a location sensor such as a Global Positioning System (GPS) location sensor. Such a sensor can detect the location, plus a speed can be detected as a rate of change of location over time. Many motion detectors output a motion signal that is indicative of the motion of the detector, and thus of the patient's body. Patient state parameters can be very helpful in narrowing down the determination of whether SCA is indeed taking place.

A WCD system made according to embodiments may thus include a motion detector. In embodiments, a motion detector can be implemented within monitoring device 180 or monitoring device 281. Such a motion detector can be made in many ways as is known in the art, for example by using an accelerometer. In this example, a motion detector 287 is implemented within monitoring device 281. A motion detector of a WCD system according to embodiments can be configured to detect a motion event. A motion event can be defined as is convenient, for example a change in motion from a baseline motion or rest, etc. In such cases, a sensed patient parameter is motion.

System parameters of a WCD system can include system identification, battery status, system date and time, reports of self-testing, records of data entered, records of episodes and intervention, and so on. In response to the detected motion event, the motion detector may render or generate, from the detected motion event or motion, a motion detection input that can be received by a subsequent device or functionality.

Environmental parameters can include ambient temperature and pressure. Moreover, a humidity sensor may provide information as to whether or not it is likely raining. Presumed patient location could also be considered an environmental parameter. The patient location could be presumed, if monitoring device 180 or 281 includes a GPS location sensor as per the above, and if it is presumed that the patient is wearing the WCD system.

Defibrillator 200 typically includes a defibrillation port 210, which can be a socket in housing 201. Defibrillation port 210 includes electrical nodes 214, 218. Leads of defibrillation electrodes 204, 208, such as leads 105 of FIG. 1, can be plugged into defibrillation port 210, so as to make electrical contact with nodes 214, 218, respectively. It is also possible that defibrillation electrodes 204, 208 are connected continuously to defibrillation port 210, instead. Either way, defibrillation port 210 can be used for guiding, via electrodes, to the wearer at least some of the electrical charge that has been stored in an energy storage module 250 that is described more fully later in this document. The electric charge will be the shock for defibrillation, pacing, and so on.

Defibrillator 200 may optionally also have a sensor port 219 in housing 201, which is also sometimes known as an ECG port. Sensor port 219 can be adapted for plugging in sensing electrodes 209, which are also known as ECG electrodes and ECG leads. It is also possible that sensing electrodes 209 can be connected continuously to sensor port 219, instead. Sensing electrodes 209 are types of transducers that can help sense an ECG signal, e.g. a 12-lead signal, or a signal from a different number of leads, especially if they make good electrical contact with the body of the patient and in particular with the skin of the patient. As with defibrillation electrodes 204, 208, the support structure can be configured to be worn by patient 282 so as to maintain sensing electrodes 209 on a body of patient 282. For example, sensing electrodes 209 can be attached to the inside of support structure 170 for making good electrical contact with the patient, similarly with defibrillation electrodes 204, 208.

Optionally a WCD system according to embodiments also includes a fluid that it can deploy automatically between the electrodes and the patient's skin. The fluid can be conductive, such as by including an electrolyte, for establishing a better electrical contact between the electrodes and the skin. Electrically speaking, when the fluid is deployed, the electrical impedance between each electrode and the skin is reduced. Mechanically speaking, the fluid may be in the form of a low-viscosity gel, so that it does not flow away, after being deployed, from the location it is released near the electrode. The fluid can be used for both defibrillation electrodes 204, 208, and for sensing electrodes 209.

The fluid may be initially stored in a fluid reservoir, not shown in FIG. 2. Such a fluid reservoir can be coupled to the support structure. In addition, a WCD system according to embodiments further includes a fluid deploying mechanism 274. Fluid deploying mechanism 274 can be configured to cause at least some of the fluid to be released from the reservoir, and be deployed near one or both of the patient locations to which electrodes 204, 208 are configured to be attached to the patient. In some embodiments, fluid deploying mechanism 274 is activated prior to the electrical discharge responsive to receiving activation signal AS from a processor 230, which is described more fully later in this document.

In some embodiments, defibrillator 200 also includes a measurement circuit 220, as one or more of its modules working together with its sensors or transducers. Measurement circuit 220 senses one or more electrical physiological signals of the patient from sensor port 219, if provided. Even if defibrillator 200 lacks sensor port 219, measurement circuit 220 may optionally obtain physiological signals through nodes 214, 218 instead, when defibrillation electrodes 204, 208 are attached to the patient. In these cases, the input reflects an ECG measurement. The patient parameter can be an ECG, which can be sensed as a voltage difference between electrodes 204, 208. In addition, the patient parameter can be an impedance, which can be sensed between electrodes 204, 208 and/or between the connections of sensor port 219 considered pairwise. Sensing the impedance can be useful for detecting, among other things, whether these electrodes 204, 208 and/or sensing electrodes 209 are not making good electrical contact with the patient's body. These patient physiological signals may be sensed when available. Measurement circuit 220 can then render or generate information about them as inputs, data, other signals, etc. As such, measurement circuit 220 can be configured to render a patient input responsive to a patient parameter sensed by a sensor. In some embodiments, measurement circuit 220 can be configured to render a patient input, such as values of an ECG signal, responsive to the ECG signal sensed by sensing electrodes 209. More strictly speaking, the information rendered by measurement circuit 220 is output from it, but this information can be called an input because it is received as an input by a subsequent device or functionality.

Defibrillator 200 also includes a processor 230. Processor 230 may be implemented in a number of ways. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 230 may include, or have access to, a non-transitory storage medium, such as memory 238 that is described more fully later in this document. Such a memory can have a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described in this document.

Processor 230 can be considered to have a number of modules. One such module can be a detection module 232. Detection module 232 can include a Ventricular Fibrillation (VF) detector. The patient's sensed ECG from measurement circuit 220, which can be available as inputs, data that reflect values, or values of other signals, may be used by the VF detector to determine whether the patient is experiencing VF. Detecting VF is useful, because VF typically results in SCA. Detection module 232 can also include a Ventricular Tachycardia (VT) detector for detecting VT, and so on.

Another such module in processor 230 can be an advice module 234, which generates advice for what to do. The advice can be based on outputs of detection module 232. There can be many types of advice according to embodiments. In some embodiments, the advice is a shock/no shock determination that processor 230 can make, for example via advice module 234. The shock/no shock determination can be made by executing a stored Shock Advisory Algorithm. A Shock Advisory Algorithm can make a shock/no shock determination from one or more ECG signals that are captured according to embodiments, and determine whether or not a shock criterion is met. The determination can be made from a rhythm analysis of the captured ECG signal or otherwise. For example, there can be shock decisions for VF, VT, etc.

In some embodiments, when the determination is to shock, an electrical charge is delivered to the patient. Delivering the electrical charge is also known as discharging and shocking the patient. As mentioned above, such can be for defibrillation, pacing, and so on.

In perfect conditions, a very reliable shock/no shock determination can be made from a segment of the sensed ECG signal of the patient. In practice, however, the ECG signal is often corrupted by electrical noise, which makes it difficult to analyze. Too much noise sometimes causes an incorrect detection of a heart arrhythmia, resulting in a false alarm to the patient. Noisy ECG signals may be handled as described in U.S. patent application Ser. No. 16/037,990, filed on Jul. 17, 2018 and since published as US 2019/0030351 A1, and also in U.S. patent application Ser. No. 16/038,007, filed on Jul. 17, 2018 and since published as US 2019/0030352 A1, both by the same applicant and incorporated herein by reference.

Processor 230 can include additional modules, such as other module 236, for other functions. In addition, if internal monitoring device 281 is indeed provided, processor 230 may receive its inputs, etc.

Defibrillator 200 optionally further includes a memory 238, which can work together with processor 230. Memory 238 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. Memory 238 is thus a non-transitory storage medium. Memory 238, if provided, can include programs for processor 230, which processor 230 may be able to read and execute. More particularly, the programs can include sets of instructions in the form of code, which processor 230 may be able to execute upon reading. Executing is performed by physical manipulations of physical quantities, and may result in functions, operations, processes, acts, actions and/or methods to be performed, and/or the processor to cause other devices or components or blocks to perform such functions, operations, processes, acts, actions and/or methods. The programs can be operational for the inherent needs of processor 230, and can also include protocols and ways that decisions can be made by advice module 234. In addition, memory 238 can store prompts for user 282, if this user is a local rescuer. Moreover, memory 238 can store data. This data can include patient data, system data and environmental data, for example as learned by internal monitoring device 281 and outside monitoring device 180. The data can be stored in memory 238 before it is transmitted out of defibrillator 200, or be stored there after it is received by defibrillator 200.

Defibrillator 200 can optionally include a communication module 290, for establishing one or more wired or wireless communication links with other devices of other entities, such as a remote assistance center, Emergency Medical Services (EMS), and so on. The communication links can be used to transfer data and commands. The data may be patient data, event information, therapy attempted, CPR performance, system data, environmental data, and so on. For example, communication module 290 may transmit wirelessly, e.g. on a daily basis, heart rate, respiratory rate, and other vital signs data to a server accessible over the internet, for instance as described in US 20140043149. This data can be analyzed directly by the patient's physician and can also be analyzed automatically by algorithms designed to detect a developing illness and then notify medical personnel via text, email, phone, etc. Module 290 may also include such interconnected sub-components as may be deemed necessary by a person skilled in the art, for example an antenna, portions of a processor, supporting electronics, outlet for a telephone or a network cable, etc.

Defibrillator 200 may also include a power source 240. To enable portability of defibrillator 200, power source 240 typically includes a battery. Such a battery is typically implemented as a battery pack, which can be rechargeable or not. Sometimes a combination is used of rechargeable and non-rechargeable battery packs. Other embodiments of power source 240 can include an AC power override, for where AC power will be available, an energy-storing capacitor, and so on. Appropriate components may be included to provide for charging or replacing power source 240. In some embodiments, power source 240 is controlled and/or monitored by processor 230.

Defibrillator 200 may additionally include an energy storage module 250. Energy storage module 250 can be coupled to the support structure of the WCD system, for example either directly or via the electrodes and their leads. Module 250 is where some electrical energy can be stored temporarily in the form of an electrical charge, when preparing it for discharge to administer a shock. In embodiments, module 250 can be charged from power source 240 to the desired amount of energy, as controlled by processor 230. In typical implementations, module 250 includes a capacitor 252, which can be a single capacitor or a system of capacitors, and so on. In some embodiments, energy storage module 250 includes a device that exhibits high power density, such as an ultracapacitor. As described above, capacitor 252 can store the energy in the form of an electrical charge, for delivering to the patient.

A decision to shock can be made responsive to the shock criterion being met, as per the above-mentioned determination. When the decision is to shock, processor 230 can be configured to cause at least some or all of the electrical charge stored in module 250 to be discharged through patient 82 while the support structure is worn by patient 82, so as to deliver a shock 111 to patient 82.

For causing the discharge, defibrillator 200 moreover includes a discharge circuit 255. When the decision is to shock, processor 230 can be configured to control discharge circuit 255 to discharge through the patient at least some of all of the electrical charge stored in energy storage module 250. Discharging can be to nodes 214, 218, and from there to defibrillation electrodes 204, 208, so as to cause a shock to be delivered to the patient. Circuit 255 can include one or more switches 257. Switches 257 can be made in a number of ways, such as by an H-bridge, and so on. Circuit 255 could also be thus controlled via processor 230, and/or user interface 280.

Defibrillator 200 can optionally include other components.

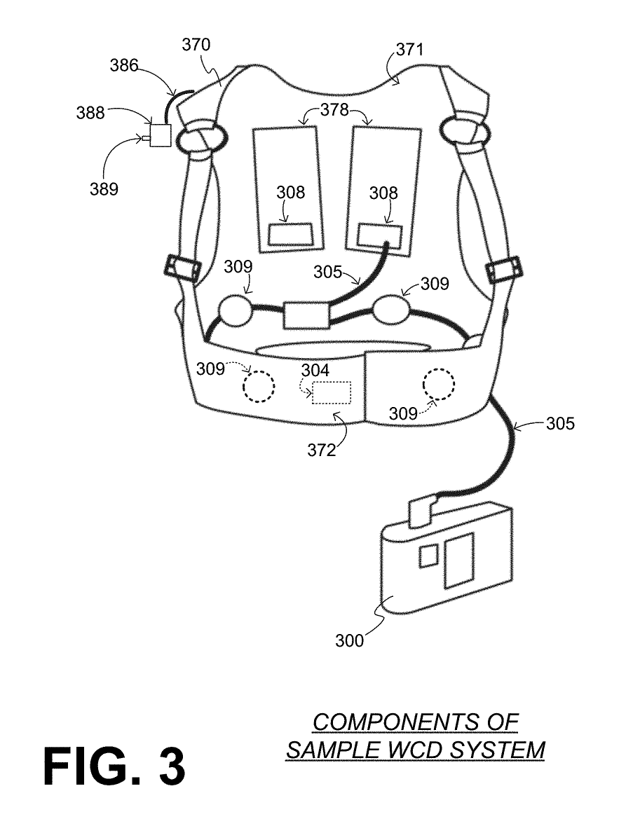

FIG. 3 is a diagram of sample embodiments of components of an WCD system. A support structure 370 includes a vest-like wearable garment. Support structure 370 has a back side 371, and a front side 372 that closes in front of the chest of the patient.

The WCD system of FIG. 3 also includes an external defibrillator 300. FIG. 3 does not show any support for external defibrillator 300, which may be carried in a purse, on a belt, by a strap over the shoulder, and so on. Wires 305 connect external defibrillator 300 to electrodes 304, 308, 309. Of those, electrodes 304, 308 are defibrillation electrodes, and electrodes 309 are ECG sensing electrodes. A therapy divert button 389 is provided on a small housing 388 which, in turn, is connected via a cable 386 to defibrillator 300, similarly to what was described with reference to FIG. 1.

Support structure 370 is configured to be worn by the ambulatory patient so as to maintain electrodes 304, 308, 309 on a body of the patient. Indeed, back defibrillation electrodes 308 are maintained in pockets 378. Of course, the inside of pockets 378 can be made with loose netting, so that electrodes 308 can contact the back of the patient, especially with the help of the conductive fluid that has been deployed. In addition, sensing electrodes 309 are maintained in positions that surround the patient's torso, for sensing ECG signals and/or the impedance of the patient.

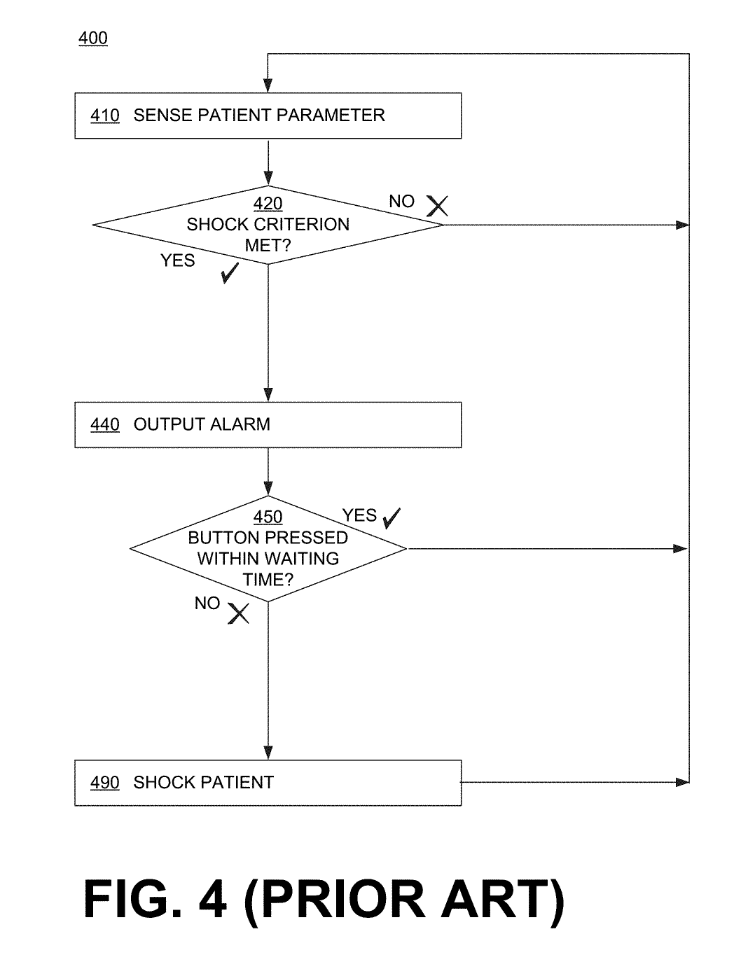

FIG. 4 is a flowchart 400 for illustrating a method about a therapy divert button of a WCD system in the prior art. According to an operation 410, a patient parameter is sensed.

According to another operation 420, it is determined whether or not a shock criterion is met. The determination is often made from the patient parameter sensed at operation 410. If not, then execution returns to operation 410. If yes, then according to another operation 440, an alarm is output to get the patient's attention and to give the patient some time to press the button.

According to another operation 450, it is determined whether or not the patient pressed the button within the given time, which is also sometimes called the waiting time. If not, then according to another operation 490, the patient is shocked as has been described. If yes, however, then execution returns to operation 410 directly and therapy has been diverted. This can mean that the processor caused no shock to be administered.

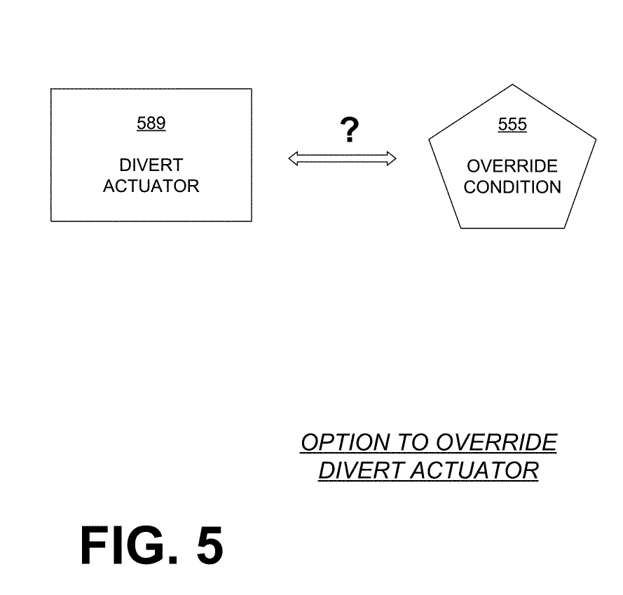

In some embodiments, actuating the divert actuator may be subject to an override condition. This is illustrated conceptually in FIG. 5 where a divert actuator 589, such as any of the previously described, may or may not be subject to an override condition 555. In embodiments where actuating divert actuator 589 is subject to override condition 555, the actuating may be disregarded by the WCD system.

The devices and/or systems mentioned in this document may perform functions, processes, acts, operations, actions and/or methods. These functions, processes, acts, operations, actions and/or methods may be implemented by one or more devices that include logic circuitry. A single such device can be alternately called a computer, and so on. It may be a standalone device or computer, such as a general-purpose computer, or part of a device that has and/or can perform one or more additional functions. The logic circuitry may include a processor and non-transitory computer-readable storage media, such as memories, of the type described elsewhere in this document. Often, for the sake of convenience only, it is preferred to implement and describe a program as various interconnected distinct software modules or features. These, along with data are individually and also collectively known as software. In some instances, software is combined with hardware, in a mix called firmware.

Moreover, methods and algorithms are described below. These methods and algorithms are not necessarily inherently associated with any particular logic device or other apparatus. Rather, they are advantageously implemented by programs for use by a computing machine, such as a general-purpose computer, a special purpose computer, a microprocessor, a processor such as described elsewhere in this document, and so on.

This detailed description may include flowcharts, display images, algorithms, and symbolic representations of program operations within at least one computer readable medium. An economy may be achieved in that a single set of flowcharts can be used to describe both programs, and also methods. So, while flowcharts describe methods in terms of boxes, they may also concurrently describe programs.

Methods are now described.

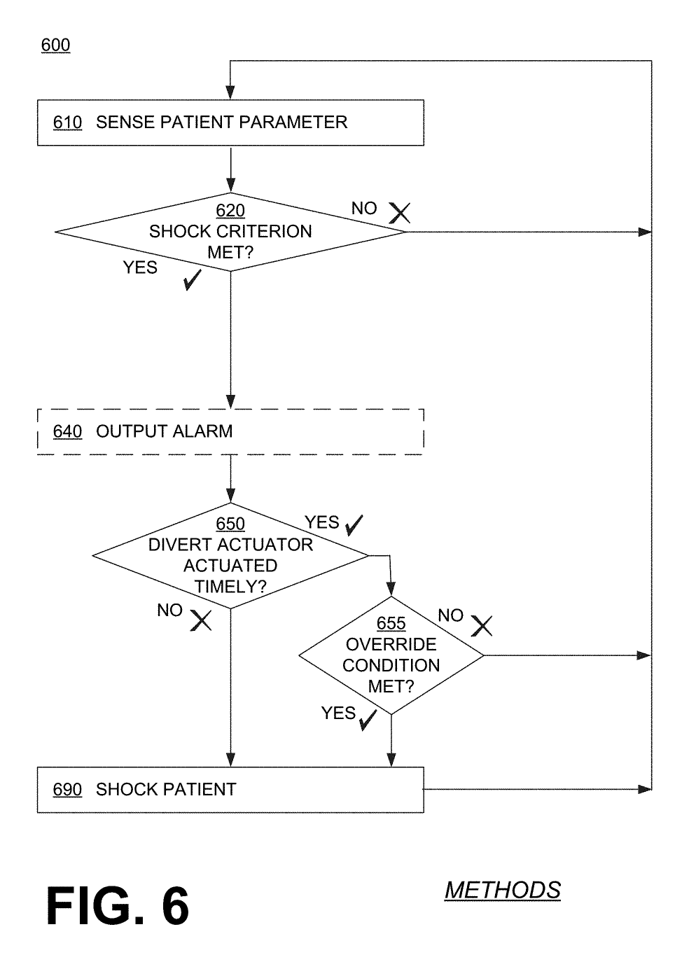

FIG. 6 shows a flowchart 600 for describing methods according to embodiments. According to an operation 610, a patient parameter may be sensed, such as an ECG, impedance, motion, breathing, pulse, circulation and so on. In some embodiments, operation 610 may be performed similarly with operation 410.

According to another, optional operation 620, it may be determined whether or not a shock criterion is met. The shock criterion may be met responsive to the patient parameter sensed at operation 610 having a value that exceeds a threshold value. In some embodiments, operation 620 may be performed similarly with operation 420.

If, at operation 620 the answer is no, then execution may return to operation 610. If the answer is yes, then according to another, optional operation 640, an alarm may be output to get the patient's attention and to give the patient some time to actuate divert actuator 589.

According to another operation 650, it may be then determined whether or not the divert actuator button has been actuated within the given time, i.e. timely. If not, then according to another operation 690, the patient is shocked as has been described. After that, execution may return to an earlier operation, such as operation 610.

If, at operation 650 the answer is yes then, according to another operation 655, it may be determined whether or not an override condition is met. The override condition may be about the divert actuator being actuated, such as override condition 555. Sample override conditions are described later in this document. If not, then execution returns to operation 610, and therapy has been diverted. This can mean that no shock was administered, or a shock was administered but the discharged charge was routed away from the patient's body.

If, at operation 650, the answer is yes then execution may proceed to operation 690, in which the operation is shocked per the above. In other words, the processor will have caused, responsive to the override condition being met at operation 655, the electrical charge to be thus discharged, even when the divert actuator is actuated.

A number of override conditions 555 are possible. Examples are now described.

In some embodiments, the override condition is met responsive to divert actuator 589 having been actuated continuously for longer than a time threshold. This may indicate that the actuator is being inadvertently pressed continuously, possibly diverting therapy when needed. This could take place in a number of ways. For instance, divert actuator 589 could be mechanically stuck. For another instance, a well-meaning but uninformed bystander may misunderstand the function of a WCD system and may want to continue pushing the button to prevent a non-responsive patient from seemingly becoming electrocuted. In such embodiments, the time threshold can have a value for a time duration that can be considered unnaturally long, such as 10 sec, 20 sec. etc.

In some embodiments, the override condition is defined in terms of when divert actuator 589 was actuated with reference to an expected sequence of events. Certain sequences of events may be regarded as unnatural, and therefore not legitimately intending to divert an imminent shock. Examples are now described.

In some embodiments, as already mentioned above, the WCD system may further have a sensor configured to sense a parameter of the patient, for example for performing operation 610. In such embodiments, the electrical charge can be configured to be thus discharged responsive to the sensed patient parameter having a value that exceeds a threshold value. The override condition can be met responsive to the divert actuator being already actuated when the parameter of the patient is sensed.

In some embodiments, as already mentioned above, the WCD system may further have a sensor configured to sense a parameter of the patient, for example for performing operation 610. The WCD system may further have output device configured to output an alarm responsive to the sensed patient parameter having a value that exceeds a threshold value. In such embodiments, the override condition can be met responsive to the divert actuator being already actuated when the alarm is output. This may be implemented from the inference that the divert actuator was actuated before it became necessary to do so, and therefore for the wrong reason.

In some embodiments, the processor of a WCD system is further configured to alternate among being in one of a plurality of states. Such may be implemented by state machines, etc. An example is now described.

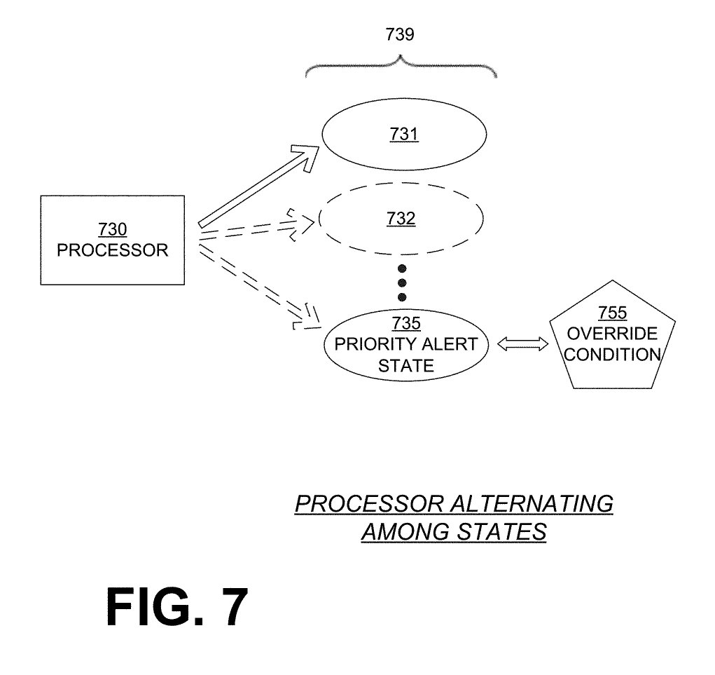

FIG. 7 is a sample state diagram for a processor 730 of a sample WCD system made according to embodiments. A set 739 has a plurality of states 731, 732, . . . 735, which are distinct from each other.

In the example of FIG. 7, processor 730 is shown as being in state 731 because a solid arrow is used to point to state 731. According to the dashed arrows, processor 730 is also capable of instead being in other states 732, 735. Processor 230, like processor 730, can be further configured to alternate among being in one of plurality 739 of states 731, 732, . . . 735. Such alternating may be triggered by events such as turning the system on and off, detection of patient states, patient inputs, shocking the patient, and so on.

These states may be given names according to their functions. For purposes of this document, state 731 is a first state, optional state 732 is a second state, state 735 is a priority alert state, and so on. In some embodiments, the stored electrical charge can be configured to be thus discharged when processor 730 is in priority alert state 735 and, in some of these embodiments, only when processor 730 is in priority alert state 735. In such embodiments, processor 730 can be configured to enter into priority alert state 735 from another one of the plurality of states of set 739 responsive to a sensed parameter of the patient having a value that exceeds a threshold value, a shock criterion being met, and so on.

Priority alert state 735 may be associated with a sample override condition 755. In some embodiments, override condition 755 can be met responsive to the divert actuator being already actuated when processor 730 enters into the priority alert state, or in other ways, for example as was described for override condition 555. Again, this may be implemented from the inference that the divert actuator was actuated before it became necessary to do so, and therefore for the wrong reason.

In some embodiments, processor 730 can be further configured to enter into another one of the plurality of states from priority alert state 735. This may be performed in a number of ways, for example responsive to override condition 755 not being met, and/or the divert actuator being actuated in a way that is deemed valid, etc. In other words, priority alert state 735 may be definitively exited if override condition 755 is not determined to not be met, and/or the divert actuator being actuated in a way that is deemed valid.

In embodiments where divert actuator 589 is a pushbutton, the result of pushing the pushbutton may vary depending on how the pushing is performed. A single click may accomplish one action, while a press-and-hold may accomplish another. In each case, it may make a difference which state processor 730 is in.

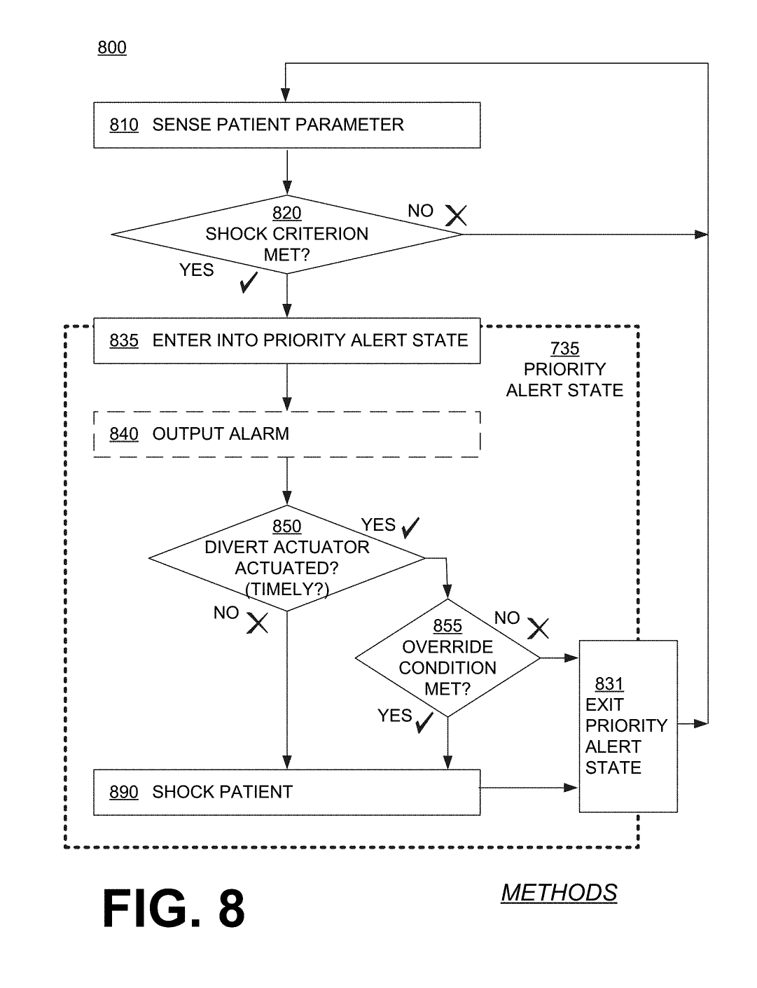

FIG. 8 shows a flowchart 800 for describing methods according to embodiments. In flowchart 800, operations 810, 820, 840, 850, 855 and 890 may be performed substantially similarly with operations 610, 620, 640, 650, 655 and 690 of flowchart 600 respectively.

If the answer at operation 820 is yes then, according to another, optional operation 835, priority alert state 735 may be entered into, from another state. And, if the answer at operation 855 is yes then, according to another, optional operation 831, priority alert state 735 may be exited, for example by going to another state. After operation 831, execution may go to a suitable operation, such as returning to operation 810.

In embodiments, when divert actuator 589 is pressed continuously, such actuation can be deemed to be accidental, inadvertent, and not legitimate. It will be appreciated that embodiments may protect the patient even in such cases, by disregarding such actuations. A sample such scenario is now described. Suppose that patient 82 suffers an event where, at operation 820 the shock criterion is met, at operation 835 priority alert state 735 is entered into, and at operation 840 an alarm is output at operation 840. Suppose further that the patient collapses in a way that improbably actuates continuously the divert actuator, after the alarm is output at operation 840. In a WCD system according to embodiments, at operation 850 the first time the answer will be yes, meaning the divert actuator has been actuated. Then, at operation 855 the first time the answer will be no, meaning the override condition is not met, and a needed shock is averted this first time, this first iteration. Then, however, at operation 831 priority alert state 735 will be exited, and the process will repeat from operation 810. The second time, however, override condition 855 will not be met because a) the actuator will have been actuated for a long time by then, and/or b) the actuator will be already actuated before operation 820, and/or c) the actuator will be already actuated before operation 840, and/or c) the actuator will be already actuated before operation 835. Therefore, in the second iteration, execution will reach process 890, and the patient will receive the needed life-saving shock.

In some embodiments, divert actuator 589 is further usable to play an instructional message to patient 82. In such embodiments, memory 238 can be configured to store data that encode an instructional message. In addition, user interface (UI) 280 may also include a UI output device for patient 82. Moreover, processor 230 can be configured to, responsive to divert actuator 589 being actuated, cause the UI output device to play the instructional message from the data stored in memory 238.

In some embodiments, the UI output device includes a microphone, and the instructional message is played as a voice message via the microphone. In some embodiments, the UI output device includes a screen, and the instructional message is played as a video message via the screen.

As was described with reference to FIG. 7, the processor of a WCD system made according to embodiments can be further configured to alternate among being in one of a plurality 739 of states. The electrical charge can be configured to be thus discharged when the processor is in priority alert state 735, or even only when the processor is in priority alert state 735. Moreover, the processor can be configured to cause the output device to play the instructional message when the processor is in first state 731. As such, actuating divert actuator 589 will cause different results depending on which state the processor is in at the time.

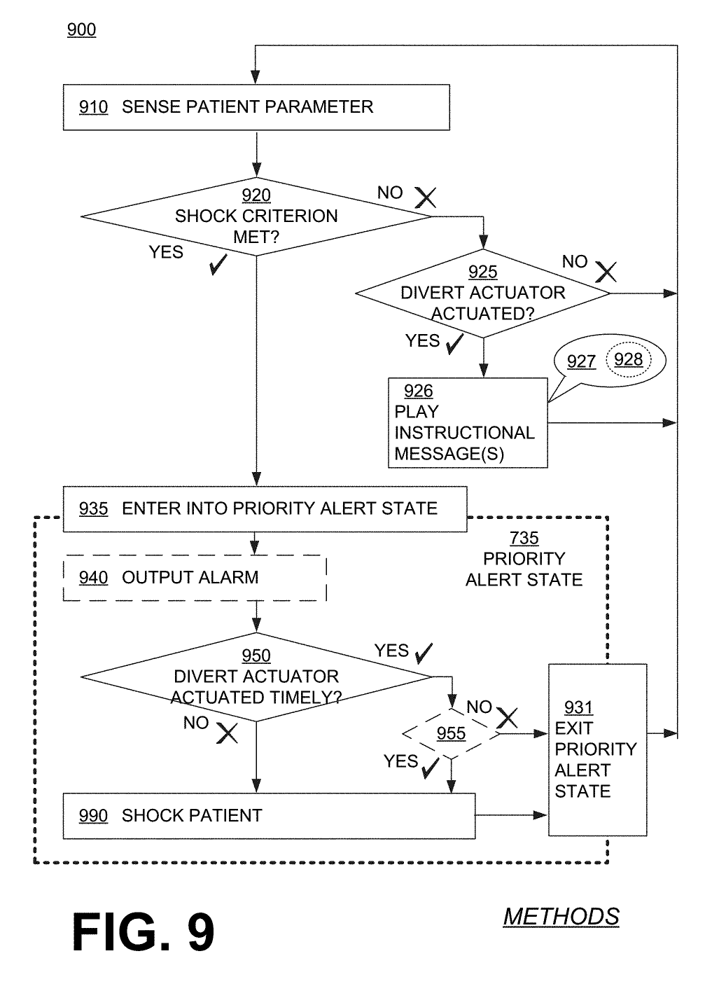

FIG. 9 shows a flowchart 900 for describing methods according to embodiments. In flowchart 900, operations 910, 920, 935, 940, 950, 955, 990 and 931 may be performed substantially similarly with respective operations 810, 820, 835, 840, 850, 855, 890 and 831 of flowchart 800 respectively. A difference is that operation 955 is optional.

Further, according to an operation 925, it may be determined whether or not the divert actuator is actuated. This determination may be made by the processor of the WCD system. It will be observed that operation 925 is rather similar to operation 950, except that it may take place when the processor is in a different state, plus time considerations may be different.

In addition, according to another operation 926, the processor may cause, responsive to the divert actuator being actuated, the UI output device to play the instructional message from the stored data. It will be appreciated that operations 925, 926 are preferably performed any time that the WCD system has perceived no crisis, such as after the yes branch of operation 920. This is why, in the example of flowchart 900, these operations 925, 926 are shown after the no branch of operation 920.

The instructional message, as played at operation 926, is depicted as a bubble 927. In embodiments, instructional message 927 has a content 928 related to the first state that the processor is in, for advising the patient accordingly as to what is happening, what they might do, and so on. Similarly, in embodiments where instructional messages can be played from different states, these messages can have contents specific to their respective states, for advising the patient accordingly.

In some embodiments, divert actuator 589 is further usable by patient 82 to initiate a long-term recording of patient data during an episode. For instance, the patient may feel discomfort, while the WCD system might not show a reaction. The patient may want to cause the WCD system to preserve its current data for a longer term, and cause that to happen by actuating divert actuator 589. This is different from any situations where a WCD system by itself automatically decides to record such data, and does so.

In such embodiments a sensor can be configured to sense a parameter of the patient, and a measurement circuit can be configured to render a patient input responsive to the sensed parameter. In addition, memory 238 can be configured to store the patient input, and erase a previously rendered patient input that has been stored for a regular time duration (RTD). Moreover, processor 230 can be configured to, responsive to divert actuator 589 being actuated, cause at least some of the patient input to remain stored in memory 238 for an extended time duration that is longer than the regular time duration. An example is now described.

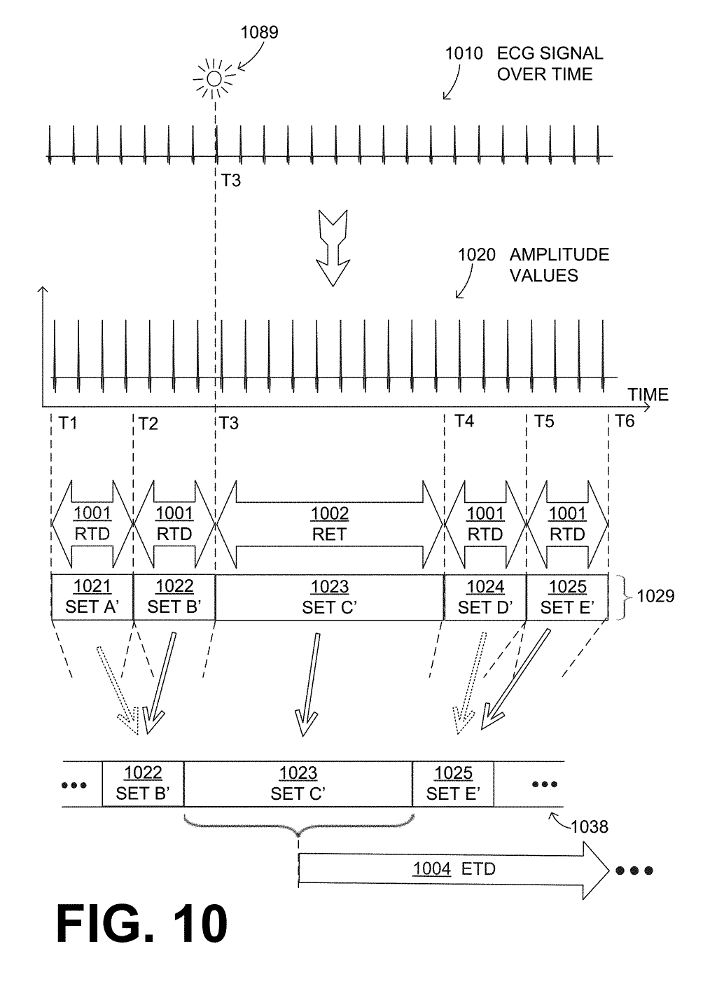

Referring to FIG. 10, a sample parameter of the patient is an ECG signal 1010, which can be sensed over time with electrodes 309. ECG signal 1010 is an electrical signal, whose amplitude is given in units of Volt. Measurement circuit 220 can be configured to render a patient input which, in the example of FIG. 10, is amplitude values 1020 over time, responsive to sensed ECG signal 1010. For values 1020, on the vertical axis there are data. And, while a horizontal time axis is used, alternately an ordinal number can be used for samples of the data values, in embodiments where measurement circuit 220 uses sampling.

If one attempted to store all amplitude values 1020 in memory 238 from say, one day, that would be impractical because a massive amount of memory would be required, and that would add to the weight that needs to be carried around by patient 82. Plus, few portions of all the stored data would be of interest.

As such, the patient input 1020 can be stored in memory 238, while erasing from memory 238 a previous and similarly-rendered patient input that has been stored for a regular time duration (RTD). In the example of FIG. 10, this is demonstrated by showing memory 1038 linearly, along a horizontal axis that might represent linearly sequential memory addresses for bytes.

A regular time duration (RTD) 1001 is shown starting from a time T1 and ending at a new time T2. The regular time duration can have a convenient value, such as 30 sec, 2 min, 5 min, etc. Between T1 and T2, there is a set A′ 1021 of the amplitude values 1020. This set A′ 1021 is initially stored in memory 1038.

Then another RTD 1001 is shown starting from time T2 and ending at a new time T3. Between T2 and T3, there is another set B′ 1022 of the amplitude values 1020. This set B′ 1022 is a patient input that becomes stored in memory 1038, while erasing set A′ 1021, which is a previous and similarly-rendered patient input that has been stored in memory 1038 for an RTD. This is why the arrow from ultimately erased set A′ 1021 towards memory 1038 is shown as dotted. The erasing is by overwriting, so as to conserve space in memory 1038.

Then, in the example of FIG. 10, at T3 there is an actuation 1089 of divert actuator 589, by the patient. Here actuation 1089 happens at exactly the end of an RTD which is not typical, but permits the diagram to be more clear for easier comprehension. Speaking of non-typical situations, a person skilled in the art will further discern that ECG 1010 shows QRS complexes with more perfect and consistent shapes and timing than is typical, even for a healthy patient. And certainly a perfect heartbeat alone would typically not provide patient 82 with cause for alarm enough for actuation 1089 but, then again, this diagram is for comprehension of storing data in the memory, not about the ECG.

Responsive to actuation 1089 at T3, at least some of the patient input is caused to remain stored in the memory for an extended time duration that is longer than the regular time duration (RTD). The amount of data that will be thus stored, long-term recorded, can be measured in time, which can be called a Recording Episode Time (RET). The RET can be longer than the RTD although that is not necessary. The RET can be adjustable, settable, etc.

In the example of FIG. 10, a RET 1002 is shown starting from time T3 and ending at a new time T4. Between T3 and T4, there is another set C′ 1023 of the amplitude values 1020. This set C′ 1023 is stored in memory 1038 in a way that it will not be erased by overwriting, and as such it is long-term recorded for an extended time duration (ETD). This is why the arrow from set C′ 1021 towards memory 1038 is shown in full lines. The extended time duration can be longer than 1 hour, and long enough until memory 1038 is downloaded, perhaps after a day of patient 82 wearing the WCD system.

In this embodiment, as preferred, set B′ 1022 is also preserved and not overwritten, although it could have been overwritten. In fact, it is preferred to have RTD 1001 be longer than shorter, because more data is thus captured prior to when the patient actuates at time T3.

In the example of FIG. 10, after T4, another RTD 1001 is shown starting from time T4 and ending at a new time T5. Between T4 and T5, there is another set D′ 1024 of the amplitude values 1020. This set D′ 1024 is stored in memory 1038, while not erasing previous set C′ 1023.

Then another RTD 1001 is shown starting from time T5 and ending at a new time T6. Between T5 and T6, there is another set E′ 1025 of the amplitude values 1020. This set E′ 1025 is stored in memory 1038, while erasing previous set D′ 1024, similarly as set A′ 1021 above was erased. This is why the arrow from ultimately erased set D′ 1024 is shown as dotted. Again, the erasing is by overwriting, so as to conserve space in memory 1038. And, if there is no actuation at that time, Set E′ 1025 will be erased afterwards, and so on.

It will be appreciated that set C′ 1023 will remain stored in memory 1038 for an extended time duration ETD 1004 that is much longer than RTD 1001 and RET 1002. That, while sets that last for RTD become routinely erased by being overwritten.

As was described with reference to FIG. 7, the processor of a WCD system made according to embodiments can be further configured to alternate among being in one of a plurality 739 of states. The electrical charge can be configured to be thus discharged when the processor is in priority alert state 735, or even only when the processor is in priority alert state 735. Moreover, the processor can be configured to cause the at least some of the patient input to remain stored in the memory for longer the regular time duration when the processor is in first state 731. As such, actuating divert actuator 589 will cause different results depending on which state the processor is in at the time.

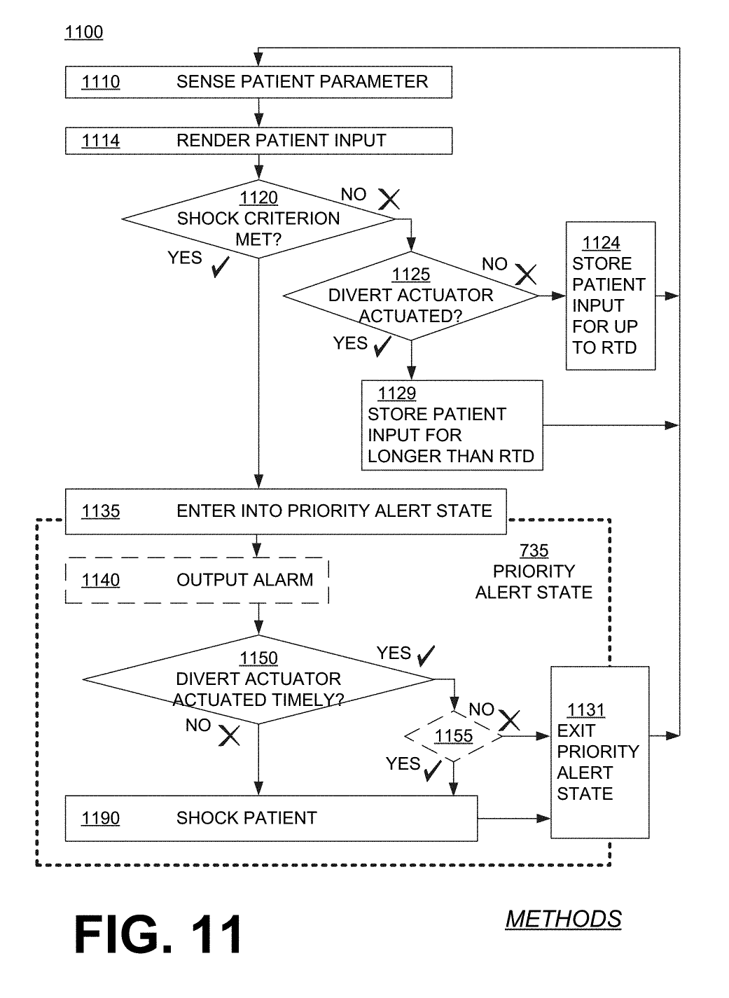

FIG. 11 shows a flowchart 1100 for describing methods according to embodiments. In flowchart 1100, operations 1110, 1120, 1125, 1135, 1140, 1150, 1155, 1190 and 1131 may be performed substantially similarly with respective operations 910, 920, 925, 935, 940, 950, 955, 990 and 931 of flowchart 900 respectively. Again, operation 1155 is optional.

In addition, according to an operation 1114, a patient input 1020 may be rendered, responsive to the parameter of the patient sensed at operation 1110. Operation 1114 may be performed, for example by measurement circuit 220. Operation 1114 may be performed also in flowcharts 600, 800, 900. Operation 1120 may be performed in view of operation 1114.

Moreover, if at operation 1125 the answer is no, then according to another operation 1124, the patient input may be stored for up to an RTD. Examples were shown for set A′ 1021 and set D′ 1024, when there was storing and then erasing after the RTD. Then execution may return to a suitable operation, such as operation 1110.

Furthermore, if at operation 1125 the answer is yes, then according to another operation 1129, at least some of the patient input can be caused to remain stored in the memory for an extended time duration that is longer than the regular time duration. An example was seen where set C′ 1023 was caused to remain stored in memory 1038 for ETD 1004 that is longer than RTD 1001.

It will be appreciated that operations 1125, 1129 are preferably performed any time that the WCD system has perceived no crisis, such as after the yes branch of operation 1120. This is why, in the example of flowchart 1100, these operations are shown after the no branch of operation 1120.

In the methods described above, each operation can be performed as an affirmative act or operation of doing, or causing to happen, what is written that can take place. Such doing or causing to happen can be by the whole system or device, or just one or more components of it. It will be recognized that the methods and the operations may be implemented in a number of ways, including using systems, devices and implementations described above. In addition, the order of operations is not constrained to what is shown, and different orders may be possible according to different embodiments. Examples of such alternate orderings may include overlapping, interleaved, interrupted, reordered, incremental, preparatory, supplemental, simultaneous, reverse, or other variant orderings, unless context dictates otherwise. Moreover, in certain embodiments, new operations may be added, or individual operations may be modified or deleted. The added operations can be, for example, from what is mentioned while primarily describing a different system, apparatus, device or method.

A person skilled in the art will be able to practice the present invention in view of this description, which is to be taken as a whole. Details have been included to provide a thorough understanding. In other instances, well-known aspects have not been described, in order to not obscure unnecessarily this description.

Some technologies or techniques described in this document may be known. Even then, however, it does not necessarily follow that it is known to apply such technologies or techniques as described in this document, or for the purposes described in this document.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In general, the present disclosure reflects preferred embodiments of the invention. The attentive reader will note, however, that some aspects of the disclosed embodiments extend beyond the scope of the claims. To the respect that the disclosed embodiments indeed extend beyond the scope of the claims, the disclosed embodiments are to be considered supplementary background information and do not constitute definitions of the claimed invention.

In this document, the phrases “constructed to”, “adapted to” and/or “configured to” denote one or more actual states of construction, adaptation and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Incorporation by reference: References and citations to other documents, such as patents, patent applications, patent publications, journals, books, papers, web contents, have been made throughout this disclosure. All such documents are hereby incorporated herein by reference in their entirety for all purposes.

Parent patent applications: Any and all parent, grandparent, great-grandparent, etc. patent applications, whether mentioned in this document or in an Application Data Sheet (“ADS”) of this patent application, are hereby incorporated by reference herein as originally disclosed, including any priority claims made in those applications and any material incorporated by reference, to the extent such subject matter is not inconsistent herewith.

Reference numerals: In this description a single reference numeral may be used consistently to denote a single item, aspect, component, or process. Moreover, a further effort may have been made in the preparation of this description to use similar though not identical reference numerals to denote other versions or embodiments of an item, aspect, component or process that are identical or at least similar or related. Where made, such a further effort was not required, but was nevertheless made gratuitously so as to accelerate comprehension by the reader. Even where made in this document, such a further effort might not have been made completely consistently for all of the versions or embodiments that are made possible by this description. Accordingly, the description controls in defining an item, aspect, component or process, rather than its reference numeral. Any similarity in reference numerals may be used to infer a similarity in the text, but not to confuse aspects where the text or other context indicates otherwise.

The claims of this document define certain combinations and subcombinations of elements, features and acts or operations, which are regarded as novel and non-obvious. The claims also include elements, features and acts or operations that are equivalent to what is explicitly mentioned. Additional claims for other such combinations and subcombinations may be presented in this or a related document. These claims are intended to encompass within their scope all changes and modifications that are within the true spirit and scope of the subject matter described herein. The terms used herein, including in the claims, are generally intended as “open” terms. For example, the term “including” should be interpreted as “including but not limited to,” the term “having” should be interpreted as “having at least,” etc. If a specific number is ascribed to a claim recitation, this number is a minimum but not a maximum unless stated otherwise. For example, where a claim recites “a” component or “an” item, it means that the claim can have one or more of this component or this item.

In construing the claims of this document, the inventor(s) invoke 35 U.S.C. § 112(f) only when the words “means for” or “steps for” are expressly used in the claims. Accordingly, if these words are not used in a claim, then that claim is not intended to be construed by the inventor(s) in accordance with 35 U.S.C. § 112(f).

1. A wearable cardioverter defibrillator (WCD) system for an ambulatory patient, comprising:
an electrode;
a support structure configured to be worn by the ambulatory patient so as to maintain the electrode on a body of the patient;
a divert actuator coupled to the support structure and configured to be actuated by the patient;
an energy storage module configured to store an electrical charge that can be discharged via the electrode through the patient so as to deliver a shock to the patient unless the divert actuator is actuated; and
a processor configured to:
determine whether or not an override condition is met, the override condition being about the divert actuator being actuated, and
cause, responsive to the override condition being met, the electrical charge to be thus discharged, even when the divert actuator is actuated.

2. The WCD system of claim 1, further comprising:
a tactile output device configured to output a confirmation vibration responsive to the divert actuator being actuated.

3. The WCD system of claim 1, in which
the override condition is met responsive to the divert actuator having been actuated continuously for longer than a time threshold.

4. The WCD system of claim 3, in which
the time threshold is 10 sec.

5. The WCD system of claim 1, further comprising:
a sensor configured to sense a parameter of the patient, and in which
the electrical charge is configured to be thus discharged responsive to the sensed patient parameter having a value that exceeds a threshold value, and
the override condition is met responsive to the divert actuator being already actuated when the parameter of the patient is sensed.

6. The WCD system of claim 1, further comprising:
a sensor configured to sense a parameter of the patient; and
an output device configured to output an alarm responsive to the sensed patient parameter having a value that exceeds a threshold value, and in which
the override condition is met responsive to the divert actuator being already actuated when the alarm is output.

7. The WCD system of claim 1, in which
the processor is further configured to:
alternate among being in one of a plurality of states distinct from each other, one of the plurality of states being a priority alert state, and
enter into the priority alert state from another one of the plurality of states responsive to a sensed parameter of the patient having a value that exceeds a threshold value,
the electrical charge is configured to be thus discharged when the processor is in the priority alert state, and
the override condition is met responsive to the divert actuator being already actuated when the processor enters into the priority alert state.

8. The WCD system of claim 7, in which
the electrical charge is configured to be thus discharged only when the processor is in the priority alert state.

9. The WCD system of claim 7, in which
the processor is further configured to enter into another one of the plurality of states from the priority alert state responsive to the override condition not being met.

10. A non-transitory computer-readable storage medium storing one or more programs which, when executed by at least one processor of a wearable cardioverter defibrillator (WCD) system for an ambulatory patient, the WCD system including an electrode, a support structure configured to be worn by the ambulatory patient so as to maintain the electrode on a body of the patient, a divert actuator coupled to the support structure and configured to be actuated by the patient, and an energy storage module configured to store an electrical charge, these one or more programs result in operations comprising:
determining whether or not the divert actuator is actuated;
determining whether or not an override condition is met, the override condition being about the divert actuator being actuated;
causing, responsive to the override condition being met, the electrical charge to be thus discharged, even when the divert actuator is actuated; and
causing the stored electrical charge to be discharged via the electrode through the patient so as to deliver a shock to the patient unless the divert actuator is actuated.

11. The medium of claim 10, in which
the WCD system further includes a tactile output device, and
when the one or more programs are executed by the at least one processor, the operations further comprise:
outputting, by the tactile output device, a confirmation vibration responsive to the divert actuator being actuated.

12. The medium of claim 10, in which
the override condition is met responsive to the divert actuator having been actuated continuously for longer than a time threshold.

13. The medium of claim 12, in which
the time threshold is 20 sec.

14. The medium of claim 10, in which
the WCD system further includes a sensor, and
when the one or more programs are executed by the at least one processor, the operations further comprise:
sensing, by the sensor, a parameter of the patient, and in which
the electrical charge is configured to be thus discharged responsive to the sensed patient parameter having a value that exceeds a threshold value, and
the override condition is met responsive to the divert actuator being already actuated when the parameter of the patient is sensed.

15. The medium of claim 10, in which
the WCD system further includes a sensor, and an output device, and
when the one or more programs are executed by the at least one processor, the operations further comprise:
sensing, by the sensor, a parameter of the patient;
outputting, by the output device, an alarm responsive to the sensed patient parameter having a value that exceeds a threshold value, and in which
the override condition is met responsive to the divert actuator being already actuated when the alarm is output.

16. The medium of claim 10, in which when the one or more programs are executed by the at least one processor, the operations further comprise:
alternating among being in one of a plurality of states distinct from each other, one of the plurality of states being a priority alert state; and
entering into the priority alert state from another one of the plurality of states responsive to a sensed parameter of the patient having a value that exceeds a threshold value, and in which
the electrical charge is configured to be thus discharged when the processor is in the priority alert state, and
the override condition is met responsive to the divert actuator being already actuated when the processor enters into the priority alert state.

17. The medium of claim 16, in which
the electrical charge is configured to be thus discharged only when the processor is in the priority alert state.

18. The medium of claim 16, in which when the one or more programs are executed by the at least one processor, the operations further comprise:
entering into another one of the plurality of states from the priority alert state responsive to the override condition not being met.

19. A method for a wearable cardioverter defibrillator (WCD) system, the WCD system including a processor, an electrode, a support structure worn by the ambulatory patient so as to maintain the electrode on a body of the patient, a divert actuator coupled to the support structure and configured to be actuated by the patient, and an energy storage module configured to store an electrical charge, the method comprising:
determining whether or not the divert actuator is actuated;
determining whether or not an override condition is met, the override condition being about the divert actuator being actuated;
causing, responsive to the override condition being met, the electrical charge to be thus discharged, even when the divert actuator is actuated; and
discharging the stored electrical charge via the electrode through the patient so as to deliver a shock to the patient unless the divert actuator is actuated.

20. The method of claim 19, in which
the WCD system further includes a tactile output device, and
further comprising:
outputting, by the tactile output device, a confirmation vibration responsive to the divert actuator being actuated.

21. The method of claim 19, in which
the override condition is met responsive to the divert actuator having been actuated continuously for longer than a time threshold.

22. The method of claim 21, in which
the time threshold is 20 sec.

23. The method of claim 19, in which
the WCD system further includes a sensor, and
further comprising:
sensing, by the sensor, a parameter of the patient, and in which
the electrical charge is configured to be thus discharged responsive to the sensed patient parameter having a value that exceeds a threshold value, and
the override condition is met responsive to the divert actuator being already actuated when the parameter of the patient is sensed.

24. The method of claim 19, in which
the WCD system further includes a sensor, and an output device, and
further comprising:
sensing, by the sensor, a parameter of the patient;
outputting, by the output device, an alarm responsive to the sensed patient parameter having a value that exceeds a threshold value, and in which
the override condition is met responsive to the divert actuator being already actuated when the alarm is output.

25. The method of claim 19, further comprising:
alternating among being in one of a plurality of states distinct from each other, one of the plurality of states being a priority alert state; and
entering into the priority alert state from another one of the plurality of states responsive to a sensed parameter of the patient having a value that exceeds a threshold value, and in which
the electrical charge is configured to be thus discharged when the processor is in the priority alert state, and
the override condition is met responsive to the divert actuator being already actuated when the processor enters into the priority alert state.

26. The method of claim 25, in which
the electrical charge is configured to be thus discharged only when the processor is in the priority alert state.

27. The method of claim 25, further comprising:
entering into another one of the plurality of states from the priority alert state responsive to the override condition not being met.

28-72. (canceled)
